(19) United States
(12) Patent Application Publication
(10) Pub. No.: US 2025/0382295 A1
(43) Pub. Date: Dec. 18, 2025

(54) SOLID STATE FORMS OF RIPRETINIB

(71) Applicant: Deciphera Pharmaceuticals, LLC, Waltham, MA (US)

(72) Inventors: Sanja Matecic Musanic, Zagreb (HR); Lara Jurkovic, Pula (HR); Valentina Travancic, Zagreb (HR)

(21) Appl. No.: 19/304,158

(22) Filed: Aug. 19, 2025

Related U.S. Application Data
(63) Continuation of application No. 17/437,552, filed on Sep. 9, 2021.
(60) Provisional application No. 62/816,547, filed on Mar. 11, 2019.
(60) Provisional application No. 62/823,039, filed on Mar. 25, 2019.
(60) Provisional application No. 62/831,816, filed on Apr. 10, 2019.
(60) Provisional application No. 62/892,045, filed on Aug. 27, 2019.

(51) Int. Cl. C07D 471/04

(52) U.S. Cl. CPC C07D 471/04

(57) ABSTRACT

The present disclosure relates to solid state forms of ripretinib, processes for preparation thereof, as well as a pharmaceutical composition including the same.

### CROSS-REFERENCE

This application is a continuation of U.S. application Ser. No. 17/437,552 filed on Sep. 9, 2021, which is a U.S. National Stage Application of International Application No. PCT/US2020/021957 filed on Mar. 11, 2020, which claims priority to U.S. Provisional Application No. 62/816,547 filed on Mar. 11, 2019, U.S. Provisional Application No. 62/823,039 filed on Mar. 25, 2019, U.S. Provisional Application 62/831,816 filed on Apr. 10, 2019, and U.S. Provisional Application No. 62/892,045 filed on Aug. 27, 2019, the contents of which are incorporated herein by reference.

### TECHNICAL FIELD

The present disclosure relates to solid state forms of ripretinib, processes for preparation thereof, as well as a pharmaceutical composition including the same.

### BACKGROUND

Ripretinib has the chemical name N-(4-bromo-5-(1-ethyl-7-(methylamino)-2-oxo-1,2-dihydro-1,6-naphthyridin-3-yl)-2-fluorophenyl)-N′-phenylurea. Ripretinib has the following chemical structure:

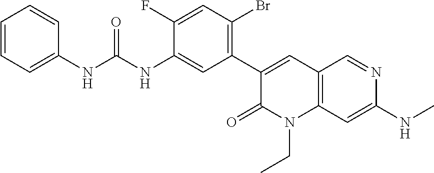 [SMILES: CCn1c(=O)c(-c2cc(NC(=O)Nc3ccccc3)c(F)cc2Br)cc2cnc(NC)cc21]

Ripretinib (DCC-2618) is an investigational, orally administered kinase switch control inhibitor being developed for the treatment of gastrointestinal stromal tumors (GIST), advanced systemic mastocytosis (ASM), gliomas, and other solid tumors driven by tyrosine-protein kinase KIT (KIT) or platelet derived growth factor alpha (PDGFRα) kinase.

Ripretinib is disclosed in U.S. Pat. No. 8,461,179.

Polymorphism, the occurrence of different crystal forms, is a property of some molecules and molecular complexes. A single compound, like ripretinib or salt thereof, may give rise to a variety of polymorphs having distinct crystal structures and physical properties like melting point, thermal behaviors (e.g. measured by thermogravimetric analysis—“TGA”, or differential scanning calorimetry—“DSC”), powder X-ray diffraction (PXRD) pattern, infrared absorption fingerprint, Raman absorption fingerprint, and solid state (¹³C-) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, improving the dissolution profile, or improving stability (polymorph as well as chemical stability) and shelf-life. These variations in the properties of different salts and solid state forms may also provide improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to use variations in the properties and characteristics of a solid active pharmaceutical ingredient for providing an improved product.

Discovering new salts, solid state forms, cocrystals and solvates of a pharmaceutical product can provide materials having desirable processing properties, such as ease of handling, ease of processing, storage stability, and ease of purification or as desirable intermediate crystal forms that facilitate conversion to other salts or polymorphic forms. New salts, polymorphic forms, cocrystals and solvates of a pharmaceutically useful compound can also provide an opportunity to improve the performance characteristics of a pharmaceutical product (dissolution profile, bioavailability, etc.). It enlarges the repertoire of materials that a formulation scientist has available for formulation optimization, for example by providing a product with different properties, e.g., a different crystal habit, higher crystallinity or polymorphic stability which may offer better processing or handling characteristics, improved dissolution profile, or improved shelf-life.

In the case of ripretinib, there is a need for a solid state form suitable for use in the pharmaceutical industry.

### SUMMARY

The present disclosure relates to solid state forms of ripretinib, to processes for preparation thereof, and to pharmaceutical compositions including these solid state forms or combinations thereof.

The present disclosure encompasses process for preparation of solid state forms of ripretinib.

The present disclosure also provides uses of the solid state forms of ripretinib for preparing other solid state forms of ripretinib, salts of ripretinib, and solid state forms thereof.

In another embodiment, the present disclosure encompasses the above described solid state forms of ripretinib for use in the preparation of pharmaceutical compositions and/or formulations, and in another embodiments for the treatment of gastrointestinal stromal tumors (GIST), advanced systemic mastocytosis (ASM), gliomas, and other solid tumors driven by tyrosine-protein kinase KIT (KIT) or platelet derived growth factor alpha (PDGFRα) kinase.

In another embodiment, the present disclosure encompasses the use of the above described solid state forms of ripretinib for the preparation of pharmaceutical compositions and/or formulations.

The present disclosure further provides pharmaceutical compositions including solid state forms of ripretinib.

In yet another embodiment, the present disclosure encompasses pharmaceutical formulations including solid state forms of ripretinib or combinations thereof and at least one pharmaceutically acceptable excipient. The pharmaceutical composition or formulation includes oral dosage forms, e.g. tablet or capsule. The present disclosure encompasses processes to prepare said pharmaceutical formulations of solid state forms of ripretinib, including combining solid state forms of ripretinib or combinations thereof, prepared according to the present disclosure, with at least one pharmaceutically acceptable excipient.

The solid state forms of ripretinib as defined herein, as well as the pharmaceutical compositions or formulations of solid state forms of ripretinib prepared according to the present disclosure, can be used as medicaments, in embodiments for the treatment of gastrointestinal stromal tumors (GIST), advanced systemic mastocytosis (ASM), gliomas, and other solid tumors driven by tyrosine-protein kinase KIT (KIT) or platelet derived growth factor alpha (PDGFRα) kinase.

The present disclosure also provides methods of treating GIST or other condition as described in the present disclosure by administering a therapeutically effective amount of solid state forms of ripretinib or combinations thereof prepared according to the present disclosure, or at least one of the above pharmaceutical compositions or formulations, to a subject suffering from GIST, or otherwise in need of the treatment.

The present disclosure also provides uses of solid state forms of the present disclosure, or at least one of the above pharmaceutical compositions or formulations, for the manufacture of a medicament for treating gastrointestinal stromal tumors (GIST), advanced systemic mastocytosis (ASM), gliomas, and other solid tumors driven by tyrosine-protein kinase KIT (KIT) or platelet derived growth factor alpha (PDGFRα) kinase.

### BRIEF DESCRIPTION OF THE FIGURES

FIG. 1 shows a powder X-ray diffraction pattern (“powder XRD” or “PXRD”) of Ripretinib form 1 prepared in Example 1;

FIG. 2 shows a PXRD of Ripretinib form 1 prepared in Example 20;

FIG. 3 shows a PXRD of Ripretinib form 3 prepared in Example 6;

FIG. 4 shows a PXRD of Ripretinib form 3 prepared in Example 19;

FIG. 5 shows a PXRD of Ripretinib amorphous form;

FIG. 6 shows a PXRD of Ripretinib form 4;

FIG. 7 shows a PXRD of Ripretinib form 5;

FIG. 8 shows a PXRD pattern of Ripretinib form 9;

FIG. 9 shows a 1H-13C CP-MAS spectra of Ripretinib form 1; and

FIG. 10 shows a 1H-13C CP-MAS spectra of Ripretinib form 3.

### DETAILED DESCRIPTION

The present disclosure relates to solid state forms of ripretinib, to processes for preparation thereof, and to pharmaceutical compositions including these solid state forms or combinations thereof.

The solid state forms of ripretinib according to the present disclosure may have advantageous properties selected from at least one of: chemical or polymorphic purity, flowability, solubility, dissolution rate, bioavailability, morphology or crystal habit, stability such as chemical stability as well as thermal and mechanical stability with respect to polymorphic conversion, stability towards dehydration and/or storage stability, a lower degree of hygroscopicity, low content of residual solvents and advantageous processing and handling characteristics such as compressibility, or bulk density.

A crystal form may be referred to herein as being characterized by graphical data “as depicted in” a Figure. Such data include, for example, powder X-ray diffractograms and solid state NMR spectra. As is well-known in the art, the graphical data potentially provides additional technical information to further define the respective solid state form (a so-called “fingerprint”) which can not necessarily be described by reference to numerical values or peak positions alone. In any event, the skilled person will understand that such graphical representations of data may be subject to small variations, e.g., in peak relative intensities and peak positions due to factors such as variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of ripretinib referred to herein as being characterized by graphical data “as depicted in” a Figure will thus be understood to include any crystal forms of ripretinib, characterized with the graphical data having such small variations, as are well known to the skilled person, in comparison with the Figure.

A solid state form (or polymorph) may be referred to herein as polymorphically pure or substantially free of any other solid state (or polymorphic) forms. As used herein in this context, the expression “substantially free of any other forms” will be understood to mean that the solid state form contains about 20% or less, about 10% or less, about 5% or less, about 2% or less, about 1% or less, or about 0% of any other forms of the subject compound as measured, for example, by PXRD. Thus, solid state forms of ripretinib and salts thereof described herein as substantially free of any other solid state forms would be understood to contain greater than about 80% (w/w), greater than about 90% (w/w), greater than about 95% (w/w), greater than about 98% (w/w), greater than about 99% (w/w), or about 100% (w/w) of the subject solid state forms of ripretinib. Accordingly, in some embodiments of the disclosure, the described solid state forms of ripretinib may contain from about 1% to about 20% (w/w), from about 5% to about 20% (w/w), or from about 5% to about 10% (w/w) of one or more other solid state forms of the same ripretinib.

The modifier “about” should be considered as disclosing the range defined by the absolute values of the two endpoints. For example, the expression “from about 2 to about 4” also discloses the range “from 2 to 4.” When used to modify a single number, the term “about” may refer to plus or minus 10% of the indicated number and includes the indicated number. For example, “about 10%” may indicate a range of 9% to 11%, and “about 1” means from 0.9-1.1.

As used herein, unless stated otherwise, PXRD peaks reported herein are preferably measured using CuK_{α} radiation, λ=1.5418 Å. Preferably, XRPD peaks reported herein are measured using CuKα radiation, λ=1.5418 Å, at a temperature of 25±3° C.

As used herein, unless stated otherwise, ¹³C CP/MAS NMR spectra employing cross-polarization were acquired using the standard pulse scheme at spinning frequency of 12 kHz. The recycle delay was 8 s and the cross-polarization contact time was 2 ms. The strength of spin-locking fields B₁(¹³C) expressed in frequency units ω1/2π=γB1 was 64 kHz, preferably at a temperature 293 K±3° C.

As used herein, the term “isolated” in reference to solid state forms of ripretinib and salts thereof of the present disclosure corresponds to solid state forms of ripretinib and salts thereof that are physically separated from the reaction mixture in which it is formed.

A thing, e.g., a reaction mixture, may be characterized herein as being at, or allowed to come to “room temperature”, often abbreviated “RT.” This means that the temperature of the thing is close to, or the same as, that of the space, e.g., the room or fume hood, in which the thing is located. Typically, room temperature is from about 20°° C. to about 30° C., or about 22° C. to about 27° C., or about 25° C. A process or step may be referred to herein as being carried out “overnight.” This refers to a time interval, e.g., for the process or step, that spans the time during the night, when that process or step may not be actively observed. This time interval is from about 8 to about 20 hours, or about 10 to about 18 hours, in embodiments about 16 hours.

As used herein, the expression “wet crystalline form” refers to a polymorph that was not dried using any conventional techniques to remove residual solvent. Examples for such conventional techniques can be, but not limited to, evaporation, vacuum drying, oven drying, drying under nitrogen flow, etc.

As used herein, the expression “dry crystalline form” refers to a polymorph that was dried using any conventional techniques to remove residual solvent. Examples of such conventional techniques can be, but are not limited to, evaporation, vacuum drying, oven drying, drying under nitrogen flow, etc.

As used herein, and unless stated otherwise, the term “anhydrous” in relation to crystalline ripretinib relates to a crystalline ripretinib which does not include any crystalline water (or other solvents) in a defined, stoichiometric amount within the crystal. Moreover, an “anhydrous” form does not contain more than 1% (w/w) of either water or organic solvents as measured for example by TGA.

The term “solvate”, as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

The amount of solvent employed in a chemical process, e.g., a reaction or a crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding methyl tert-butyl ether (MTBE) (1.5 v/v) to a 100 ml reaction mixture would indicate that 150 mL of MTBE was added.

As used herein, the term “reduced pressure” refers to a pressure of about 10 mbar to about 50 mbar.

As used herein the term non-hygroscopic in relation to crystalline ripretinib refers to less than 0.2% (w/w) of water absorption after 24h exposure to 25° C./80% RH, determined according to European Pharmacopoeia 10.0, chapter 01/2008:51100. Water can be for example atmospheric water.

The present disclosure includes solid state forms of ripretinib (free base). The present disclosure further includes crystalline forms of ripretinib. In embodiments, the crystalline forms according to the present invention may be anhydrous forms. In other embodiments, crystalline forms according to the present invention may be solvates including hydrates. In other embodiments, crystalline forms according to the invention may be mixed solvates. In yet further embodiments there is provided a solid state form of ripretinib which is amorphous.

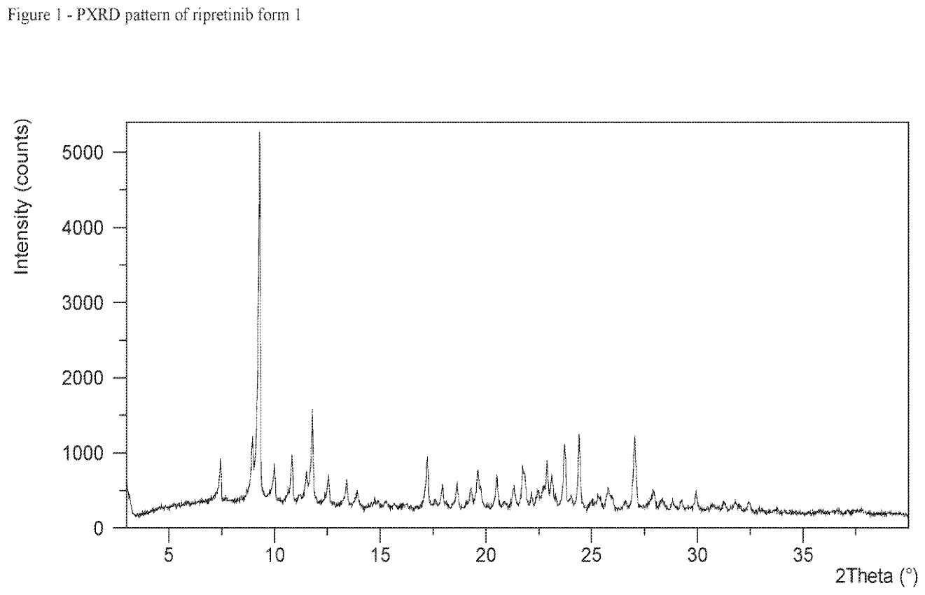

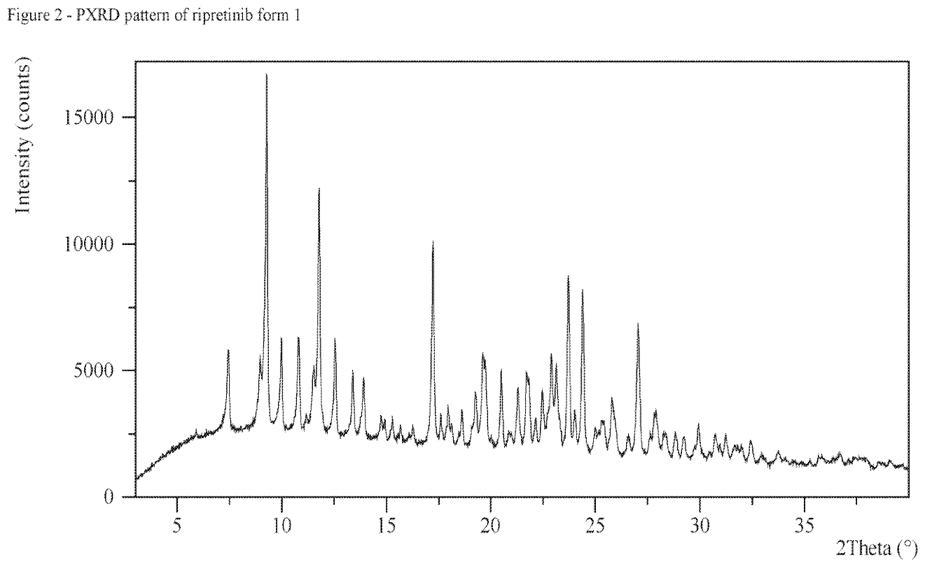

The present disclosure further includes a solid state form of ripretinib designated as Form 1. Form 1 of ripretinib can be characterized by data selected from one or more of the following: a PXRD pattern having peaks at 9.3, 11.8, 17.2, 24.4 and 27.0 degrees 2-theta±0.2 degrees 2-theta; a PXRD pattern as depicted in FIG. 1; or combinations of these data. Alternatively, Form 1 of ripretinib can be characterized by data selected from one or more of the following: a PXRD pattern having peaks at 9.3, 11.8, 17.2, 24.4 and 27.0 degrees 2-theta±0.2 degrees 2-theta; a PXRD pattern as depicted in FIG. 2; or combinations of these data.

Form 1 of ripretinib may be further characterized by the PXRD pattern having peaks at 9.3, 11.8, 17.2, 24.4 and 27.0 degrees 2-theta±0.2 degrees 2-theta, and also having one, two, three, four or five additional peaks at 7.4, 10.8, 18.6, 20.5 and 23.7 degrees 2-theta±0.2 degrees 2-theta.

Alternatively, Form 1 of ripretinib may be characterized by a PXRD pattern having peaks at 7.4, 9.3, 10.8, 11.8, 17.2, 18.6, 20.5, 23.7, 24.4 and 27.0 degrees 2-theta±0.2 degrees 2-theta.

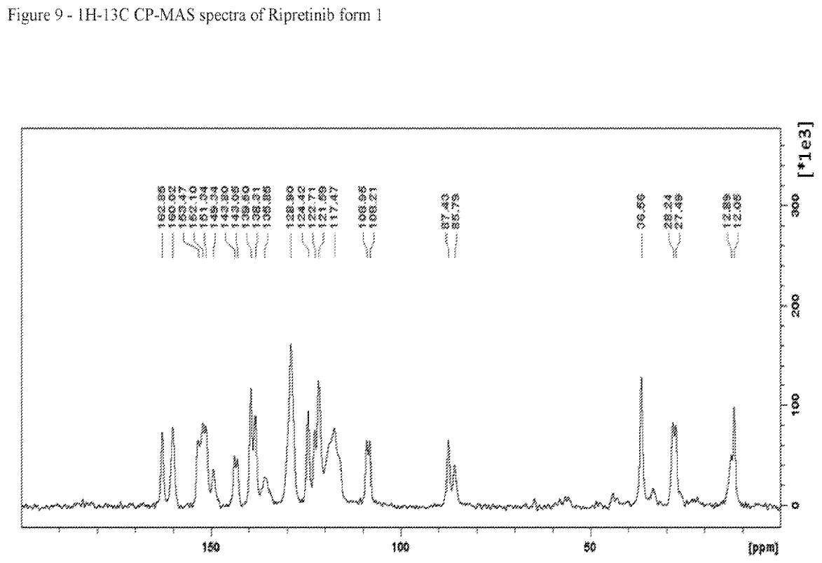

In any embodiment of the present invention Form 1 of ripretinib may alternatively or additionally be characterized by a solid state 13C NMR spectrum having peaks at 162.9, 160.0, 139.5, 128.9, 121.6±0.2 ppm. Form 1 of ripretinib may alternatively or additionally be characterized by a solid state 13C NMR spectrum having the following chemical shift absolute differences from a reference peak at 87.4 ppm±0.2 ppm of 75.5, 72.6, 52.1, 41.5, 34.2±0.1 ppm. In any embodiment of the present invention, Form 1 of ripretinib may alternatively or additionally be characterized by a solid state 13C NMR spectrum substantially as depicted in FIG. 9.

Form 1 of ripretinib may be characterized by each of the above characteristics alone or by all possible combinations, e.g., by a PXRD pattern having peaks at 9.3, 11.8, 17.2, 24.4 and 27.0 degrees 2-theta±0.2 degrees 2-theta and a PXRD pattern as depicted in FIG. 1.

Form 1 of ripretinib according to any of the above embodiments may be an anhydrous form. In any embodiment of the present invention, Form 1 may be non-hygroscopic.

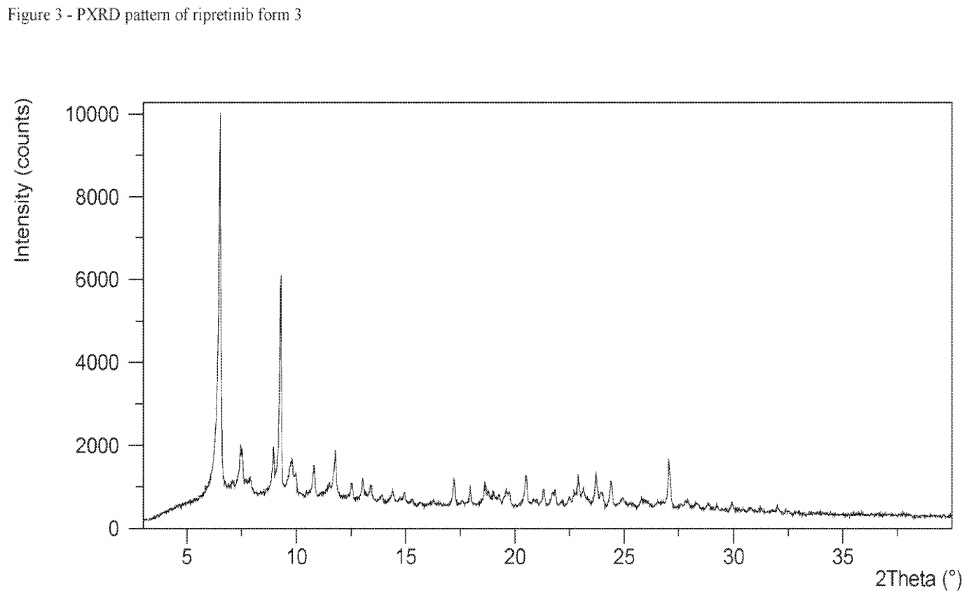

The present disclosure further includes a solid state form of ripretinib designated as Form 3. Form 3 of ripretinib can be characterized by data selected from one or more of the following: a PXRD pattern having peaks at 6.5, 9.3, 9.8, 11.8 and 13.1 degrees 2-theta±0.2 degrees 2-theta; a PXRD pattern as depicted in FIG. 3; or combinations of these data.

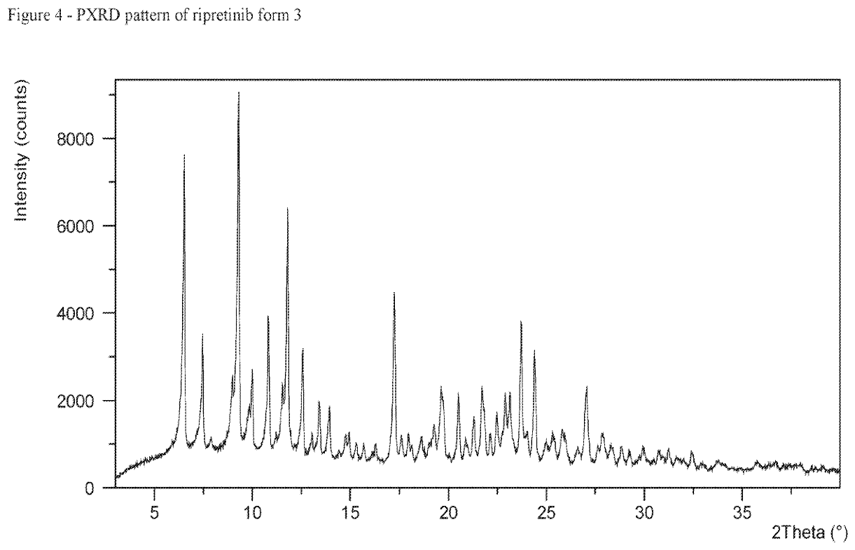

In any embodiment of the present invention Form 3 of ripretinib may alternatively or additionally be characterized by data selected from one or more of the following: a PXRD pattern having peaks at 6.5, 9.3, 9.8, 11.8 and 13.1 degrees 2-theta±0.2 degrees 2-theta; a PXRD pattern as depicted in FIG. 4; or combinations of these data.

Form 3 of ripretinib may be further characterized by the PXRD pattern having peaks at 6.5, 9.3, 9.8, 11.8 and 13.1 degrees 2-theta±0.2 degrees 2-theta, and also having one, two, three, four or five additional peaks at 10.8, 14.4, 17.2, 20.5 and 27.0 degrees 2-theta±0.2 degrees 2-theta.

Alternatively, Form 3 of ripretinib may be characterized by a PXRD pattern having peaks at 6.5, 9.3, 9.8, 10.8, 11.8, 13.1, 14.4, 17.2, 20.5 and 27.0 degrees 2-theta±0.2 degrees 2-theta.

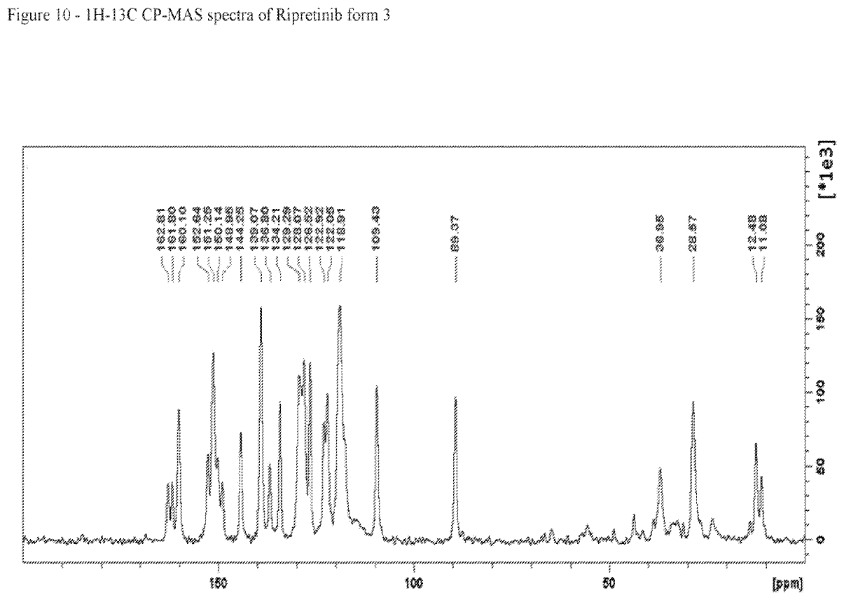

In any embodiment of the present invention Form 3 of ripretinib may alternatively or additionally be characterized by a solid state 13C NMR spectrum having peaks at 160.1, 151.3, 139.1, 118.9, 109.4±0.2 ppm. Form 3 of ripretinib may alternatively or additionally be characterized by a solid state 13C NMR spectrum having the following chemical shift absolute differences from a reference peak at 89.4 ppm±0.2 ppm of 70.7, 61.9, 49.7, 29.5, 20.0±0.1 ppm. In any embodiment of the present invention, Form 3 of ripretinib may alternatively or additionally be characterized by a solid state 13C NMR spectrum substantially as depicted in FIG. 10.

Form 3 of ripretinib may be characterized by each of the above characteristics alone or by all possible combinations, e.g., by a PXRD pattern having peaks at 6.5, 9.3, 9.8, 11.8 and 13.1 degrees 2-theta±0.2 degrees 2-theta and a PXRD pattern as depicted in FIG. 3.

Form 3 of ripretinib according to any of the above embodiments may be a hydrate form.

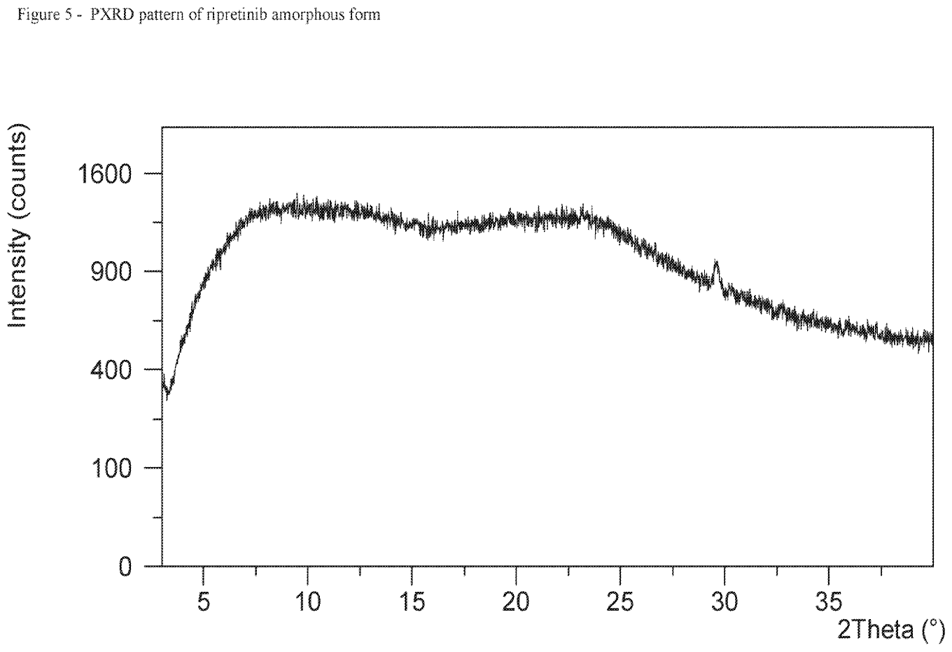

The present disclosure further includes a solid state form of ripretinib designated as amorphous form. Amorphous form of ripretinib can be characterized by a PXRD pattern as depicted in FIG. 5.

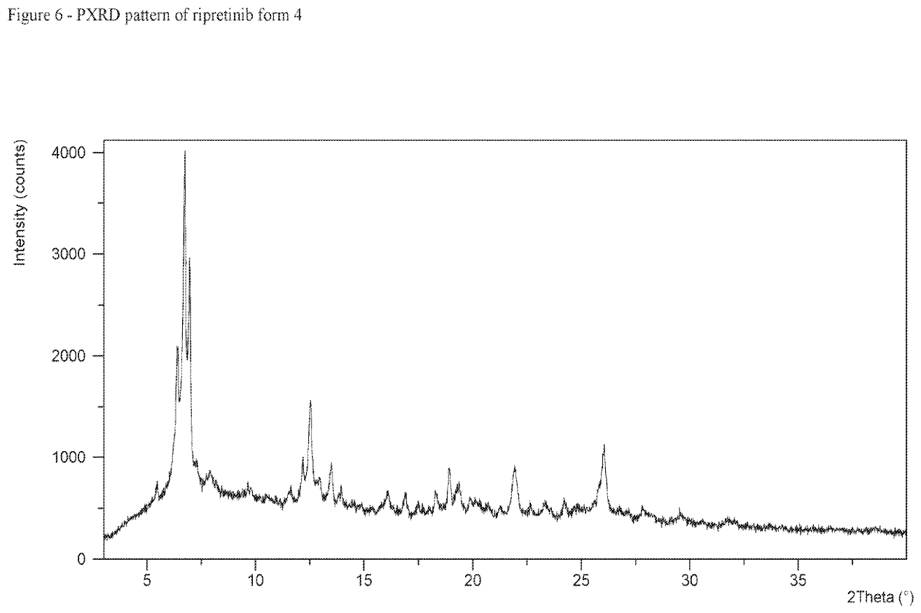

The present disclosure further includes a solid state form of ripretinib designated as Form 4. Form 4 of ripretinib can be characterized by data selected from one or more of the following: a PXRD pattern having peaks at 6.7, 12.5, 18.9, 21.9 and 26.0 degrees 2-theta±0.2 degrees 2-theta; a PXRD pattern as depicted in FIG. 6; or combinations of these data.

Form 4 of ripretinib may be further characterized by the PXRD pattern having peaks at 6.7, 12.5, 18.9, 21.9 and 26.0 degrees 2-theta±0.2 degrees 2-theta, and also having one, two, three, four or five additional peaks at 7.0, 11.6, 13.5, 16.1 and 24.2 degrees 2-theta±0.2 degrees 2-theta.

Alternatively, Form 4 of ripretinib may be characterized by a PXRD pattern having peaks at 6.7, 7.0, 11.6, 12.5, 13.5, 16.1, 18.9, 21.9, 24.2 and 26.0 degrees 2-theta±0.2 degrees 2-theta.

Form 4 of ripretinib may be characterized by each of the above characteristics alone or by all possible combinations, e.g., by a PXRD pattern having peaks at 6.7, 12.5, 18.9, 21.9 and 26.0 degrees 2-theta±0.2 degrees 2-theta and a PXRD pattern as depicted in FIG. 6.

Form 4 of ripretinib according to any of the above embodiments may be a DMSO/H₂O solvate.

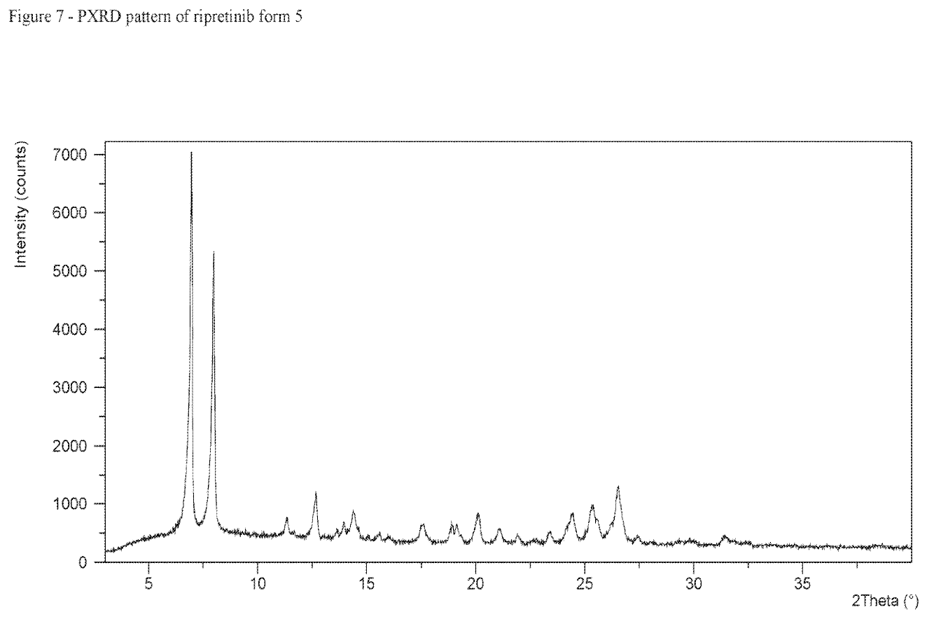

The present disclosure further includes a solid state form of ripretinib designated as Form 5. Form 5 of ripretinib can be characterized by data selected from one or more of the following: a PXRD pattern having peaks at 7.0, 8.0, 12.7, 20.1 and 26.5 degrees 2-theta±0.2 degrees 2-theta; a PXRD pattern as depicted in FIG. 7; or combinations of these data.

Form 5 of ripretinib may be further characterized by the PXRD pattern having peaks at 7.0, 8.0, 12.7, 20.1 and 26.5 degrees 2-theta±0.2 degrees 2-theta, and also having one, two, three, four or five additional peaks at 11.4, 14.4, 17.6, 24.4 and 25.3 degrees 2-theta±0.2 degrees 2-theta.

Alternatively, Form 5 of ripretinib may be characterized by a PXRD pattern having peaks at 7.0, 8.0, 11.4, 12.7, 14.4, 17.6, 20.1, 24.4, 25.3 and 26.5 degrees 2-theta±0.2 degrees 2-theta.

Form 5 of ripretinib may be characterized by each of the above characteristics alone or by all possible combinations, e.g., by a PXRD pattern having peaks at 7.0, 8.0, 12.7, 20.1 and 26.5 degrees 2-theta±0.2 degrees 2-theta and a PXRD pattern as depicted in FIG. 7.

Form 5 of ripretinib according to any of the above embodiments may be an acetic acid solvate.

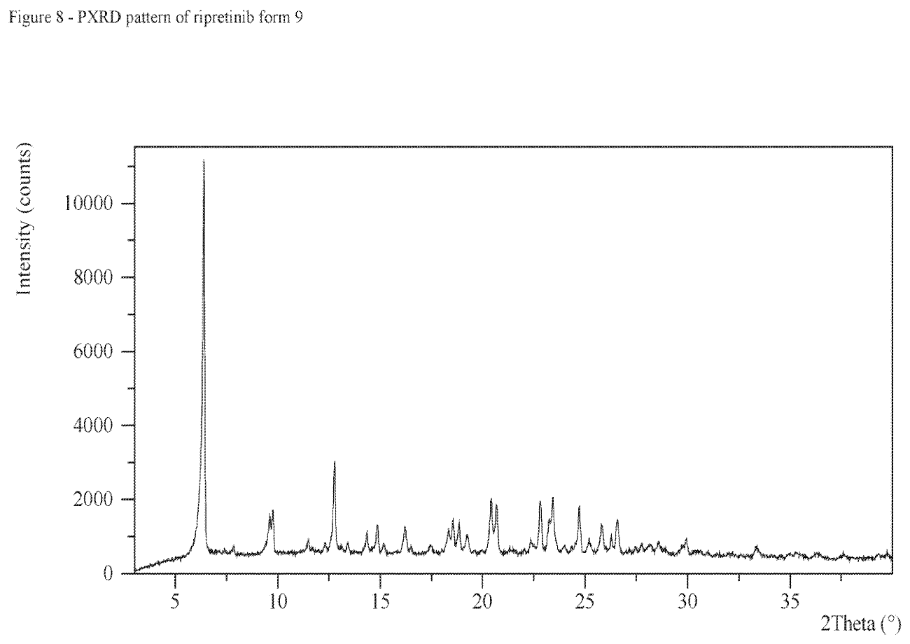

The present disclosure further includes a solid state form of ripretinib designated as Form 9. Form 9 of ripretinib can be characterized by data selected from one or more of the following: a PXRD pattern having peaks at 6.4, 12.8, 16.2, 20.7 and 24.7 degrees 2-theta±0.2 degrees 2-theta; a PXRD pattern as depicted in FIG. 8; or combinations of these data.

Form 9 of ripretinib may be further characterized by the PXRD pattern having peaks at 6.4, 12.8, 16.2, 20.7 and 24.7 degrees 2-theta±0.2 degrees 2-theta, and also having one, two, three, four or five additional peaks at 18.9, 20.4, 22.8, 23.4, and 33.4 degrees 2-theta±0.2 degrees 2-theta.

Alternatively, Form 9 of ripretinib may be characterized by a PXRD pattern having peaks at 6.4, 12.8, 16.2, 18.9, 20.4, 20.7, 22.8, 23.4, 24.7 and 33.4 degrees 2-theta±0.2 degrees 2-theta.

Form 9 of ripretinib may be characterized by each of the above characteristics alone or by all possible combinations, e.g., by a PXRD pattern having peaks at 6.4, 12.8, 16.2, 20.7 and 24.7 degrees 2-theta±0.2 degrees 2-theta and a PXRD pattern as depicted in FIG. 8.

Form 9 of ripretinib according to any of the above embodiments may be a DMSO solvate.

The present disclosure also provides the use of the solid state forms of ripretinib of the present disclosure for preparing different solid state forms of ripretinib, salts of ripretinib and solid state forms thereof.

The present disclosure further encompasses processes for preparing the solid state forms of ripretinib of the present disclosure. The disclosure further includes processes for preparing different solid state forms of ripretinib or salts of ripretinib and solid state forms thereof. The process includes preparing at least one of the solid state forms of ripretinib of the present disclosure, and converting it to different solid state forms of ripretinib or salts of ripretinib and solid state forms thereof. The conversion can be done, for example, by a process including reacting at least one of the obtained solid state forms of ripretinib with an appropriate acid to obtain ripretinib acid addition salt.

In another embodiment the present disclosure encompasses the above described solid state forms of ripretinib for use in the preparation of pharmaceutical compositions and/or formulations, in embodiments for the treatment of GIST.

In another embodiment the present disclosure encompasses the use of the above described solid state forms of ripretinib for the preparation of pharmaceutical compositions and/or formulations.

The present disclosure further provides pharmaceutical compositions including the solid state forms of ripretinib of the present disclosure.

In yet another embodiment, the present disclosure encompasses pharmaceutical formulations including solid state forms of ripretinib of the present disclosure, and at least one pharmaceutically acceptable excipient. The pharmaceutical formulations according to the present disclosure may be for oral administration in a form of a tablet or a capsule.

Pharmaceutical formulations of the present invention contain any one or a combination of the solid state forms of ripretinib of the present disclosure. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present invention include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present disclosure, the active ingredient and any other solid excipients may be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present disclosure can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, the most preferred route of the present disclosure is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the invention, within either a hard or soft shell. The shell can be made from gelatin and optionally contain a plasticizer such as glycerin and sorbitol, and an opacifying agent or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present invention can comprise any of the aforementioned blends and granulates that were described with reference to tableting, but they are not subjected to a final tableting step.

In further embodiments, a pharmaceutical formulation of ripretinib is formulated for administration to a mammal, such as a human. Ripretinib can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The present disclosure encompasses a process to prepare said formulations of solid state forms of ripretinib by combining the solid state forms prepared according to the present disclosure and at least one pharmaceutically acceptable excipient.

Solid state forms of ripretinib as defined herein, as well as the pharmaceutical compositions or formulations of ripretinib can be used as medicaments, in embodiments for the treatment of GIST.

The present disclosure also provides a method of treating of GIST, by administering a therapeutically effective amount of the solid state forms of ripretinib prepared according to the present disclosure, or at least one of the above pharmaceutical compositions or formulations, to a subject suffering from GIST, or otherwise in need of the treatment.

The present disclosure also provides the use of solid state forms of ripretinib, or at least one of the above pharmaceutical compositions or formulations for the manufacture of a medicament for treating GIST.

Having described the solid state forms of ripretinib with reference to certain exemplary embodiments, other embodiments will become apparent to one skilled in the art from consideration of the specification. The disclosure is further illustrated by reference to the following examples describing in detail the preparation of the composition and methods of use of the disclosure. It will be apparent to those skilled in the art that many modifications, both to materials and methods, may be practiced without departing from the scope of the disclosure.

### Analytical Methods

### Powder X-Ray Diffraction Pattern (“PXRD”) Method:

Sample is powdered in a mortar and pestle and applied directly on a silicon plate holder. The X-ray powder diffraction pattern was measured with Philips X'Pert PRO X-ray powder diffractometer, equipped with Cu irradiation source=1.54184 Å (Ångström), X'Celerator (2.022° 2θ) detector. Scanning parameters: angle range: 3-40 deg., step size 0.0167, time per step 37 seconds, continuous scan.

The described peak positions were determined without using silicon powder as an internal standard in an admixture with the sample measured.

¹³C Solid State Nuclear Magnetic Resonance (“ss-NMR” or 13C Solid State NMR) Method

Solid state NMR spectra were measured at 11.7 T using a Bruker Avance III HD 500 US/WB NMR spectrometer (Karlsruhe, Germany, 2013). The ¹³C CP/MAS NMR spectra employing cross-polarization were acquired using the standard pulse scheme at spinning frequency of 12 kHz. The recycle delay was 8 s and the cross-polarization contact time was 2 ms. The strength of spin-locking fields B₁(¹³C) expressed in frequency units ω1/2π=γB1 was 64 kHz.

The ¹³C NMR scale was referenced to a-glycine (176.03 ppm). Frictional heating of the spinning samples was offset by active cooling, and the temperature calibration was performed with Pb(NO₃)₂. The NMR spectrometer was completely calibrated and all experimental parameters were carefully optimized prior the investigation. Magic angle was set using KBr during standard optimization procedure and homogeneity of magnetic field was optimized using adamantane sample (resulting line-width at half-height Dn1/2 was less than 3.5 Hz at 250 ms of acquisition time).

### EXAMPLES

Ripretinib can be prepared according to any known method, for example the procedure described in U.S. Pat. No. 8,461,179.

### Example 1: Preparation of Ripretinib Form 1

A suspension of ripretinib (20 mg) in acetonitrile (10 mL) was heated to the boiling point. The suspension was cooled down to room temperature. The solid was isolated by vacuum filtration. The obtained solid corresponds to ripretinib form 1 as confirmed by PXRD.

### Example 2: Preparation of Ripretinib Form 1

A suspension of ripretinib (200 mg) in acetonitrile (100 mL) was heated to the boiling point. The suspension was cooled down to room temperature. The solid was isolated by vacuum filtration. The obtained solid corresponds to ripretinib form 1 as confirmed by PXRD.

### Example 3: Preparation of Ripretinib Form 1

A suspension of ripretinib (20 mg) in different solvents (triethylamine, amyl acetate, amyl alcohol, i-butanol, i-butyl acetate, n-butyl acetate, sec-butyl acetate, tert-butyl acetate, cumene, 1,2-dichloroethene, 1,2-dichloroethane, cyclomethylethyl ether, diethyl malonate, diethoxymethane, diisopropyl ether, ethyl acetoacetate, ethylbenzene, 1-propanol, i-propyl acetate, n-propyl acetate, 1,2-propylene carbonate, 3-pentanone, m-xylene) (10 mL) was heated to the boiling point. The suspension was cooled down to room temperature. The solid was isolated by vacuum filtration. The obtained solid corresponds to ripretinib form 1 as confirmed by PXRD.

### Example 4: Preparation of Ripretinib Form 3

A suspension of Ripretinib (20 mg) in different solvents (10 mL, water; mixture of 3% acetone in water; or mixture of 1% methanol in water) was heated to the boiling point. The suspension was cooled down to room temperature. The solid was isolated by vacuum filtration. The obtained solid corresponds to ripretinib form 3 as confirmed by PXRD.

### Example 5: Preparation of Ripretinib Form 3

A suspension of Ripretinib (200 mg) in mixture of 3% acetone in water (100 mL) was heated to the boiling point. The suspension was cooled down to room temperature. The solid was isolated by vacuum filtration. The obtained solid corresponds to ripretinib form 3 as confirmed by PXRD.

### Example 6: Preparation of Ripretinib Form 3

A suspension of Ripretinib (400 mg) in mixture of 3% acetone in water (200 mL) was heated to the boiling point. The suspension was cooled down to room temperature. The solid was isolated by vacuum filtration. The obtained solid corresponds to ripretinib form 3 as confirmed by PXRD.

### Example 7: Preparation of Amorphous Ripretinib

Ripretinib (500 mg) was subjected to milling in ball mill (agate jar, 2 agate balls ϕ=7 mm, frequency: 25 Hz, time: 6 hours). The obtained solid corresponds to amorphous ripretinib as confirmed by PXRD.

### Example 8: Preparation of Amorphous Ripretinib

Ripretinib (4 grams) was subjected to milling in ball mill (zirconium oxide jar, 7 zirconium oxide balls, ϕ=9 mm, frequency: 800 rpm, time: 6 hours). The obtained solid corresponds to amorphous ripretinib as confirmed by PXRD.

### Example 9: Preparation of Ripretinib Form 4

Ripretinib (500 mg) was dissolved in dimethyl sulfoxide (30 mL). The obtained clear solution was filtered and was frozen using a liquid nitrogen bath within 10 minutes, and lyophilized for 72 hours (temperature −40° C., pressure 270-330 μbar). The obtained solid corresponds to ripretinib form 4 as confirmed by PXRD.

### Example 10: Preparation of Ripretinib Form 5

A suspension of Ripretinib (200 mg) in mixture of acetic acid (60 mL) was heated to the boiling point. The solution was cooled down to the room temperature. The solid was isolated by vacuum filtration. The obtained solid corresponds to ripretinib form 5 as confirmed by PXRD.

### Example 11: Preparation of Ripretinib Form 1

A suspension of Ripretinib (50 mg, amorphous form) and acetone or methanol (2 mL) was stirred in closed clear glass vial at 450 rpm for 1 day at room temperature. The solid was isolated by vacuum filtration. Ripretinib Form 1 was obtained as confirmed by PXRD.

### Example 12: Preparation of Ripretinib Form 1

Ripretinib (120 mg, amorphous form) was dissolved in NMP (N-methylpyrrolidone) (11 ml) at room temperature, and water (previously cooled to 0-5° C.) was added dropwise in solution (44 ml). The crystallization was momentary. Obtained solid was isolated by vacuum filtration. Ripretinib Form 1 was obtained as confirmed by PXRD.

### Example 13: Preparation of Ripretinib Form 1

Ripretinib (305 mg, amorphous form) was dissolved in solvent mixture DMSO:Water (16 ml, 9:1 volume ratio,) at 50° C., and water (previously cooled to 0-5° C.) was added dropwise in solution (64 ml), and additionally stirred at 450 rpm for 1 hour. Obtained solid was isolated by vacuum filtration. Ripretinib Form 1 was obtained as confirmed by PXRD.

### Example 14: Preparation of Ripretinib Form 1

Ripretinib (314 mg, amorphous form) was dissolved in solvent mixture DMSO:Water (35 ml, 9:1 volume ratio) at 80° C., and water (water temperature approximately 25° C.) (105 ml) was added dropwise in solution, and additionally stirred at 450 rpm for 1 hour. Obtained solid was isolated by vacuum filtration. Ripretinib Form 1 was obtained as confirmed by PXRD.

### Example 15: Preparation of Ripretinib Form 1

Ripretinib (1960 mg, amorphous form) was dissolved in solvent mixture DMSO:Water (300 ml, 9:1 volume ratio,) at 60° C. (in 1 L reactor) and water (water temperature approximately 25° C.) (300 ml) was added dropwise in solution, and additionally stirred (mechanical stirrer) at 250 rpm for 1 hour. Obtained solid was isolated by vacuum filtration. Ripretinib Form 1 was obtained as confirmed by PXRD.

### Example 16: Preparation of Ripretinib Form 9

Ripretinib (305 mg, amorphous form) was dissolved in solvent mixture DMSO:Water (60 ml, 9:1 volume ratio,) at room temperature, and water (previously cooled to 0-5° C.) (180 ml) was added dropwise in solution. Solution was crystallized after 13 ml of added water, and additionally stirred at 450 rpm for 1 hour. Obtained solid was isolated by vacuum filtration. Ripretinib Form 9 was obtained as confirmed by PXRD.

### Example 17: Preparation of Ripretinib Form 9

Ripretinib (329 mg, amorphous form) was dissolved in solvent mixture DMSO:Water (35 ml, 9:1 volume ratio,) at 40° C., and water (water temperature approximately 25° C.) (105 ml) was added dropwise in solution, and additionally stirred at 450 rpm for 1 hour. Obtained solid was isolated (302 mg, Yield: 91.7%) by vacuum filtration. Ripretinib Form 9 was obtained as confirmed by PXRD.

### Example 18: Preparation of Ripretinib Form 3

A suspension of Ripretinib (50 mg, form 9) and solvent mixture acetone: Water (2 ml, 1:9 volume ratio,) was stirred in closed clear glass vial at 350 rpm for 3 days at room temperature. The solid was isolated by vacuum filtration. Ripretinib Form 3 was obtained as confirmed by PXRD.

### Example 19: Preparation of Ripretinib Form 3

A suspension of Ripretinib (620 mg, form 9) and solvent mixture Acetone: Water (20 ml, 1:9 volume ratio,) was stirred at 500 rpm for 90 min at 70° C. The solid was isolated by vacuum filtration. Ripretinib Form 3 was obtained as confirmed by PXRD.

### Example 20: Preparation of Ripretinib Form 1

Ripretinib (307 mg, amorphous form) was dissolved in solvent mixture DMSO:Water (37 ml, 9: 1volume ratio) at 60° C., and water was added dropwise in solution (74 ml), and additional stirred by 450 rpm for 1 hour. Solution was crystallized after 13 ml of water was added. Obtained solid was isolated by vacuum filtration. Ripretinib Form 1 was obtained as confirmed by PXRD.

### Example 21: Preparation of Ripretinib Form 1

A suspension of Ripretinib (50 mg, amorphous form) and acetone (2 mL) or methanol (2 mL) was stirred in closed clear glass vial at 450 rpm for 1 day at room temperature. The solid was isolated by vacuum filtration. Ripretinib Form 1 was obtained as confirmed by PXRD.

### Example 22: Preparation of Amorphous Ripretinib

Ripretinib (500 mg, form 1) was subjected to milling in ball mill (agate jar, 2 agate balls ϕ=7 mm, frequency: 25 Hz, time: 6 hours). The obtained solid corresponds to amorphous ripretinib as confirmed by PXRD.

1. A solid-state form of ripretinib, wherein the solid-state form is amorphous.

2. The solid-state form of claim 1, wherein the solid-state form is characterized by a PXRD pattern substantially as depicted in FIG. 5.

3. The solid-state form of claim 1, wherein the solid-state form is a free base.

4. The solid-state form of claim 1, wherein the solid-state form is a salt.

5. The solid state form of claim 2, wherein the solid-state form is a salt.

6. The solid state form of claim 2, wherein the solid-state form is a free base.

7. The solid-state form of claim 1, wherein the solid-state form is substantially free of any other solid-state forms.

8. The solid-state form of claim 2, wherein the solid-state form is substantially free of any other solid-state forms.

9. The solid-state form of claim 3, wherein the solid-state form is substantially free of any other solid-state forms.

10. The solid-state form of claim 4, wherein the solid-state form is substantially free of any other solid-state forms.

11. The solid-state form of claim 5, wherein the solid-state form is substantially free of any other solid-state forms.

12. The solid-state form of claim 6, wherein the solid-state form is substantially free of any other solid-state forms.

13. The solid-state form of claim 5, wherein the solid-state form contains 5% or less of any other solid-state form.

14. The solid-state form of claim 5, wherein the solid-state form contains 2% or less of any other solid-state form.

15. The solid-state form of claim 5, wherein the solid-state form contains 1% or less of any other solid-state form.

16. The solid-state form of claim 5, wherein the solid-state form is substantially pure.

17. A process for preparing an amorphous solid-state form of ripretinib, comprising ball milling a solid-state form of ripretinib.

18. The solid-state form of claim 1, obtained according to the process of claim 10.

19. A method of treating gastrointestinal stromal tumors (GIST), advanced systemic mastocytosis (ASM), gliomas, and other solid tumors driven by tyrosine-protein kinase KIT (KIT) or platelet derived growth factor alpha (PDGFRα) kinase in a patient in need thereof, comprising administering to the patient the solid-state form of claim 1.

20. The method of claim 12 for treating gastrointestinal stromal tumors (GIST).
